(19) United States
(12) Patent Application Publication
(10) Pub. No.: US 2026/0053458 A1
(43) Pub. Date: Feb. 26, 2026

(54) RADIOGRAPHIC IMAGING APPARATUS AND RADIOGRAPHIC IMAGING SYSTEM

(71) Applicant: KONICA MINOLTA, INC., Tokyo (JP)

(72) Inventors: Yuuichi MARUTA, Tokyo (JP); Nobuyuki MIYAKE, Kanagawa (JP)

(73) Assignee: KONICA MINOLTA, INC., Tokyo (JP)

(21) Appl. No.: 19/300,381

(22) Filed: Aug. 14, 2025

(30) Foreign Application Priority Data
Aug. 23, 2024 (JP) 2024-141993

(51) Int. Cl. A61B 6/00; A61B 6/42

(52) U.S. Cl. CPC A61B 6/542; A61B 6/4266

(57) ABSTRACT

An image processing apparatus according to the present disclosure is a radiographic imaging apparatus that generates a moving image including a plurality of frames by repeatedly performing an accumulation period in which charges based on radiation emitted from a radiation source are accumulated and a reading period in which the accumulated charges are read. The radiographic imaging apparatus includes: an output section that outputs information for irradiation control of the radiation from the radiation source based on dose information of the emitted radiation; and an image controller that directly or indirectly determines, based on the dose information, at least one of a start timing and an end timing of the reading period in at least one of a current frame and a next frame of the current frame based on the radiation corresponding to the acquired dose information.

### CROSS REFERENCE TO RELATED APPLICATIONS

The entire disclosure of Japanese Patent Application No. 2024-141993 filed on Aug. 23, 2024, is incorporated herein by reference in its entirety.

### BACKGROUND

### Technological Field

The present disclosure relates to a radiographic imaging apparatus and a radiographic imaging system.

### Description of Related Art

Description of the Related Art A radiographic imaging system that generates a radiographic image by detecting radiation transmitted through a subject using a radiation detection element has been widely used.

An example of a radiographic imaging apparatus used in such a radiographic imaging system is a flat panel detector (FPD). The FPD has a structure in which a plurality of radiation detection elements are arranged in a matrix. Each radiation detection element is provided with a switch that controls signal reading. When the subject is irradiated with radiation from the radiation source, charges corresponding to the detected amount of radiation are accumulated in the radiation detection element. In this state, the switches are individually controlled to sequentially read charges from the radiation detection element, thereby generating a radiographic image.

Some of such radiographic imaging systems can generate a moving image. The radiation moving image is generated by, for example, the following step. The radiation source repeatedly emits radiation pulses at a cycle of a predetermined number of times per unit time over a predetermined period. In parallel with this, the FPD accumulates charges generated in accordance with the dose of radiation received by the radiation detection element of the FPD through the subject over a predetermined period (accumulation period), and then repeatedly reads the amount of accumulated charges as a signal value in a cycle of a predetermined number of times per unit time, thereby generating a frame image (still image) for each unit time based on the accumulated charge amount. That is, a radiation moving image is generated by sequentially arranging frame images whose imaging times are different from each other by a pulse period.

In order to appropriately generate a radiation moving image, it is necessary to synchronize an irradiation period in which the radiation source emits radiation and an accumulation period of charges in each radiation detection element of the FPD. As an example of the technique for synchronizing the irradiation period and the accumulation period, Japanese Unexamined Patent Publication No. 2013-94174 discloses a technique in which clocks of an X-ray generating apparatus and an image collecting apparatus are synchronized with each other in a state where no irradiation is performed, a difference between the clocks is constantly monitored, and when a difference is determined to have occurred, the difference is corrected.

### SUMMARY

In a case where a moving image is generated in such a radiographic imaging system, a series of irradiation of radiation from the radiation generating apparatus to the subject and processing of reading charges from each radiation detection element are alternately performed. The radiographic imaging apparatus can generate a moving image by repeating the operation of performing reading processing after a series of radiation irradiations ends.

In the case of generating a moving image, it is desirable that the radiation irradiation cycle and the radiation irradiation time of the radiation source be constant throughout a series of imaging operations. However, the irradiation cycle and the irradiation time are not constant in some cases. In such a case, the irradiation period and the accumulation period cannot be synchronized with each other, and the image quality of the generated moving image may be degraded.

An object of the present disclosure is to provide a radiographic imaging apparatus and a radiographic imaging system that can accurately synchronize an irradiation period by a radiation source and an accumulation period of charges due to detection of radiation in a case of generating a moving image.

To achieve at least one of the abovementioned objects, according to an aspect of the present disclosure, there is provided a radiographic imaging apparatus that generates a moving image including a plurality of frames by repeatedly executing an accumulation period in which charges based on radiation emitted from a radiation source are accumulated and a reading period in which the accumulated charges are read. The radiographic imaging apparatus including a hardware processor that executes: outputting, based on dose information of the emitted radiation, information for controlling emission of the radiation from the radiation source; and directly or indirectly determining, based on the dose information, at least one of a start timing and an end timing of the reading period in at least one of a current frame and a next frame of the current frame based on the radiation corresponding to the acquired dose information.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a block diagram illustrating an overall configuration of a radiation diagnostic system;

FIG. 2 is a block diagram illustrating an equivalent circuit of an FPD cassette;

FIG. 3 is a block diagram illustrating an example of the configuration of a controller;

FIGS. 4A and 4B are diagrams for describing a method of determining the reading start timing by the image controller;

FIG. 5 is a block diagram illustrating a functional configuration of a radiation control apparatus; and

FIGS. 6A, 6B, and 6C are diagrams illustrating examples of setting screens for inputting a frame rate or a dose per unit time (a tube voltage (kV value), a tube current (mA), an irradiation time (sec), a mAs value, or the like) before image capturing.

### DETAILED DESCRIPTION OF EMBODIMENTS

Hereinafter, one or more embodiments of the present invention will be described with reference to the drawings. However, the scope of the invention is not limited to the disclosed embodiments.

In the following, embodiments of the present disclosure will be described with reference to the drawings.

### First Embodiment

First, the first embodiment will be described.

### Configuration of Radiographic Imaging System 100

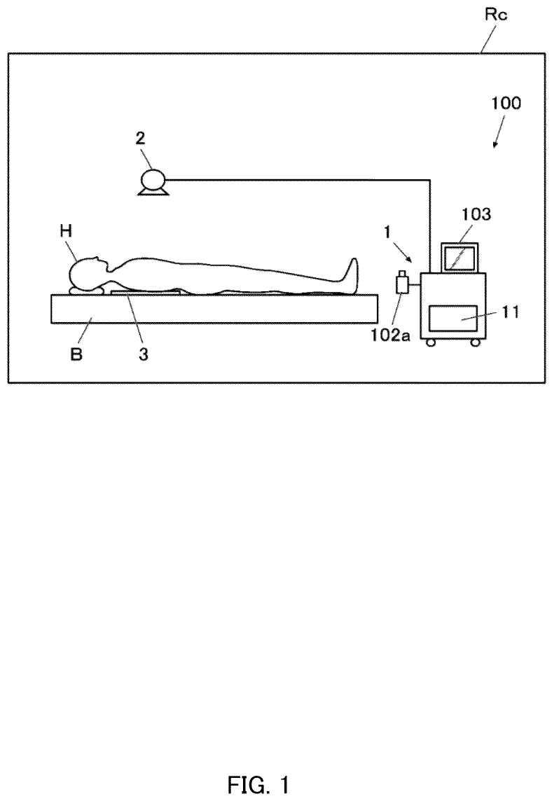

FIG. 1 is a diagram illustrating an example of an overall configuration of a radiographic imaging system 100 according to an embodiment of the present disclosure. The radiographic imaging system 100 is a system for capturing a radiographic image by irradiating a subject with radiation, and includes a radiation control apparatus 1, a radiation source 2, and a flat panel detector (FPD) cassette 3. The FPD cassette 3 is an example of the radiographic imaging apparatus of the present disclosure.

In the example illustrated in FIG. 1, the radiation control apparatus 1 is a movable medical cart having wheels. In the example illustrated in FIG. 1, the radiographic imaging system 100 is brought into and used in an operating room, an intensive care unit, a hospital room Rc, or the like. The FPD cassette 3 is disposed, for example, between a bed B and a subject H lying on the bed B. The radiation source 2 emits radiation under the control of the radiation control apparatus 1. The FPD cassette 3 outputs a detection signal based on the radiation that has passed through the subject. The radiation control apparatus 1 generates a radiographic image based on the detection signals.

Note that the present disclosure is not limited to the aspect illustrated in FIG. 1, and for example, each component may be installed and used in an inspection room or the like.

In the present disclosure, the radiographic imaging system 100 can generate a moving image of the subject H. The radiographic imaging system 100 repeatedly irradiates the subject H with pulsed radiation such as X-rays at predetermined time intervals (pulse irradiation) in response to an imaging operation. Here, a moving image is generated by repeatedly executing an accumulation period in which the FPD cassette 3 is irradiated with radiation from the radiation source 2 and charges are accumulated in the radiation detection element of the FPD cassette 3, and a reading period in which the charges accumulated in the radiation detection element are read. Each of the plurality of images constituting the moving image is referred to as a frame image. One frame image is generated through one accumulation period and one reading period. In the present specification, a period including one accumulation period and one reading period may be referred to as a frame (one frame). That is, one frame is a period corresponding to one frame image.

Each of the apparatuses constituting the radiographic imaging system 100 will be described below.

### FPD Cassette 3

The FPD cassette 3 is a radiographic imaging apparatus compatible with moving image capturing. In the present embodiment, the FPD cassette 3 is an indirect imaging apparatus that converts the emitted radiation into light having another wavelength, such as visible light, with a scintillator and obtains image data with a radiation detection element. The present disclosure is not limited thereto, and the radiographic imaging apparatus of the present disclosure may be a direct-type imaging apparatus that directly detects radiation with a radiation detection element without using a scintillator.

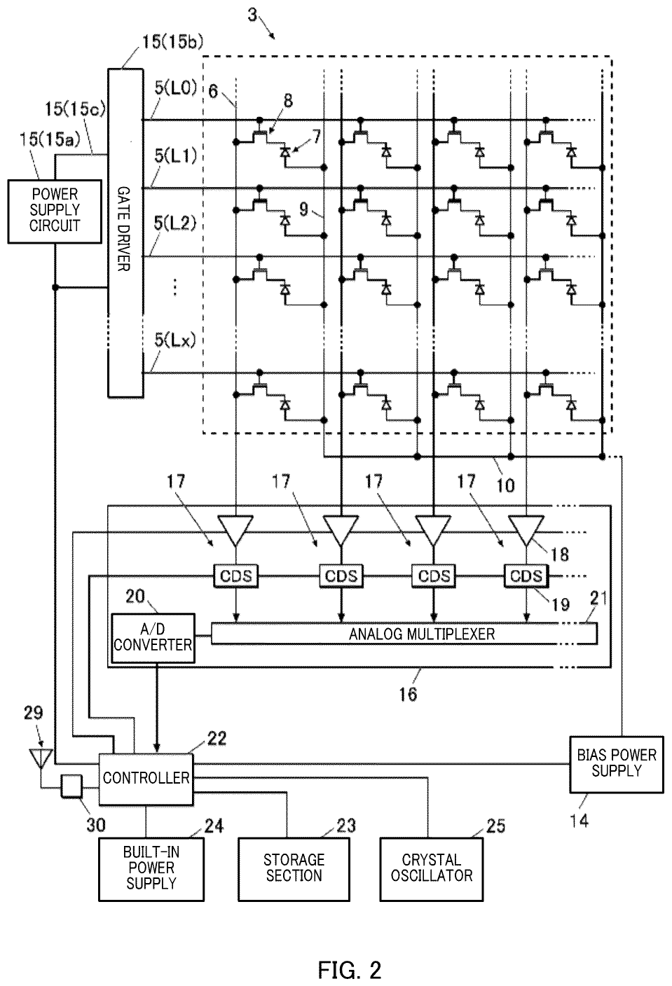

FIG. 2 is a block diagram illustrating an equivalent circuit of the FPD cassette 3. As illustrated in FIG. 2, the FPD cassette 3 includes a plurality of radiation detection elements 7, a scanning drive section 15, a reading IC 16, a controller 22, a storage section 23, a built-in power supply 24, a timing device 25, and a wireless communication section 30.

The plurality of radiation detection elements 7 are arranged in a matrix on the substrate. Each radiation detection element 7 accumulates charges corresponding to the dose of the emitted radiation. A bias line 9 is connected to each radiation detection element 7. The bias line 9 is connected to a bias power source 14 via a connection line 10. With such a structure, a reverse bias voltage is applied from the bias power supply 14 to each radiation detection element 7.

A thin film transistor (TFT) 8 is connected to each radiation detection element 7 as a switch element. The TFT 8 is connected to the signal line 6. Some of the radiation detection elements 7 arranged as the radiation detection elements 7 individually operate as radiation detection elements that detect the amount of incident radiation during a period in which exposure control is performed, that is, during radiation irradiation. In the present specification, such an element is referred to as a dose detection element. A dose detection value to be described later is a value obtained from the dose detection element.

The scanning drive section 15 includes a power supply circuit 15a, a gate driver 15b, and a wire 15c. In the scanning drive section 15, an on-voltage and an off-voltage supplied from the power supply circuit 15a via the wire 15c are switched by the gate driver 15b and applied to the lines L0 to Lx of the scanning line 5. When an on-voltage is applied to each TFT 8 via the scanning line 5, the TFT 8 is turned on to release charges accumulated in the radiation detection element 7 to the signal line 6. In addition, when an off-voltage is applied to each TFT 8 via the scanning line 5, each TFT 8 is turned off to interrupt the conduction between the radiation detection element 7 and the signal line 6, thereby accumulating charges generated in the radiation detection element 7 in the radiation detection element 7. Each of the radiation detection element 7 (each TFT 8) forms a pixel (pixel).

Reading IC 16 reads charges emitted from each radiation detection element 7 to the signal line 6. The reading IC 16 includes a plurality of reading circuits 17, amplifier circuits 18, correlated double sampling (CDS) circuits 19, an A/D converter 20, and a multiplexer 21.

In the reading processing, when charges are released from the radiation detection element 7, the amplifier circuit 18 outputs a voltage value corresponding to the amount of charges that have flowed into the reading circuit 17 via the signal line 6. The CDS 19 reads the voltage value output from the amplifier circuit 18 as a detection signal having an analog value, and outputs the detection signal to the downstream. The detection signals having analog values are sequentially transmitted to the A/D converter 20 via the multiplexer 21. The A/D converter 20 sequentially converts the detection signals having analog values into digital values. The detection signals having digital values are sequentially stored in the storage section 23.

The controller 22 is composed of a computer in which a central processing unit (CPU), a read only memory (ROM), a random access memory (RAM), an input/output interface, and the like are connected to a bus, a field programmable gate array (FPGA), or the like. The controller 22 may be formed of a dedicated control circuit. The controller 22 is connected to a storage section 23 including a static RAM (SRAM), a synchronous DRAM (SDRAM), a NAND flash memory, and the like.

The controller 22 controls operations of the scanning drive section 15 and the reading circuit 17. Thus, the controller 22 can cause each radiation detection element 7 to accumulate charges corresponding to the amount of radiation, and can cause the reading circuit 17 to read out the accumulated charges. The controller 22 determines the start timing of reading charges from each radiation detection element 7 based on a dose detection value obtained from a dose detection element that is a part of the radiation detection element 7.

The configuration that the controller 22 has for controlling the operation of the scanning drive section 15 and the reading circuit 17 will be described later.

The wireless communication section 30 performs wireless communication with the radiation control apparatus 1 via the antenna 29 based on the control by the controller 22. Since the communication between the radiation control apparatus 1 and the FPD cassette 3 is performed in a wireless manner, it is not necessary to connect the radiation control apparatus 1 and the FPD cassette 3 in a wired manner by a cable or the like at the time of capturing a radiographic image, which is convenient.

The wireless communication section 30 transmits, for example, the detection signal read by the reading circuit 17 to the radiation control apparatus 1, and transmits a control signal for controlling the radiation source 2 via the radiation control apparatus 1. Further, the wireless communication section 30 receives a control signal for the operation of the FPD cassette 3 from the radiation control apparatus 1.

The built-in power supply 24 supplies necessary electric power to the scanning drive section 15, the reading circuits 17, the controller 22, the storage section 23, the bias power supply 14, and the like.

The timing device 25 is, for example, a crystal oscillator that oscillates due to the piezoelectric effect. The controller 22 can measure the time with accuracy based on the number of oscillations of the crystal oscillator. The timing device 25 is an example of a timing device according to the present disclosure. Note that in the present disclosure, the timing device is not limited to a crystal oscillator, and may be any device that can measure time with accuracy.

Here, a description will be given of a configuration that the controller 22 has for controlling the operation of the scanning drive section 15 and the reading circuit 17.

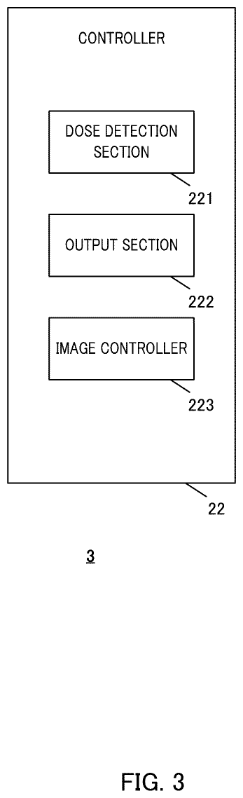

FIG. 3 is a block diagram illustrating an example of the configuration of the controller 22. As illustrated in FIG. 3, the controller 22 includes a dose detection section 221, an output section 222, and an image controller 223.

The dose detection section 221 detects the dose detection value of the radiation emitted from the radiation source 2 toward the FPD cassette 3 in the accumulation period, by using the detection values read from some of the plurality of radiation detection elements 7 in the accumulation period. The dose detection value is an example of the dose information of the present disclosure. The dose detection section 221 detects a dose detection value by, for example, sampling detection values at two or more different times within one accumulation period. Note that the accumulation period is a period in which the FPD cassette 3 is irradiated with radiation from the radiation source 2 via the subject and charges are accumulated in the radiation detection elements 7 of the FPD cassette 3.

The output section 222 performs automatic exposure control for the radiation source 2 based on the dose detection value. Automatic exposure control refers to control such that radiation is emitted at an appropriate exposure amount in each frame in order to obtain a high-quality radiographic image. The automatic exposure control is an example of the irradiation control of the present disclosure. The output section 222 notifies the radiation control apparatus 1 that the dose detection value exceeds a predetermined first threshold value, for example, at a timing when the dose detection value exceeds the first threshold value. When the radiation control apparatus 1 controls the radiation source 2 to stop irradiation, automatic exposure control is performed. Note that the method of automatic exposure control is not limited to the above-described example and may be implemented, for example, by the output section 222 periodically transmitting the dose detection value to the radiation control apparatus 1 and the radiation control apparatus 1 comparing the dose detection value with the threshold each time and controlling the radiation source 2 to stop irradiation. Alternatively, the output section 222 may directly control the radiation source 2 to stop the subsequent radiation irradiation without passing through the radiation control apparatus 1.

In the present disclosure, a known technique can be appropriately adopted as the automatic exposure control executed by the output section 222.

The image controller 223 generates control signal for the scanning drive section 15 and the reading circuit 17 based on the imaging condition and the dose detection value. More specifically, the control signal for determining the start timing of the reading period in the current frame and subsequent frames includes at least information such as the length of the accumulation period in each frame, the start timing of the reading period, and the length of the reading period. The reading period is a period in which the emission of radiation from the radiation source 2 is stopped and charges are read from each radiation detection element 7 of the FPD cassette 3. In the following description, the start timing of the reading period may be referred to as a reading start timing.

The present disclosure is characterized in that the image controller 223 controls the reading start timing in the reading circuit 17 using a dose detection value in the accumulation period. The dose detection value is generated by the dose detection section 221 for the automatic exposure control for the radiation source 2 by the output section 222. With this characteristic, the irradiation end timing of radiation in the radiation source 2 and the reading start timing in the reading circuit 17 are synchronized with each other with accuracy. Therefore, the radiographic imaging system 100 can generate a radiation moving image with high image quality.

In this way, the image controller 223 controls the reading start timing in the reading circuit 17 and consequently in the FPD cassette 3 using the dose detection value for automatic exposure control, and thus the following effects are obtained.

As a first effect, it is possible to extend a period during which highly accurate synchronization can be maintained, in other words, a time available for imaging during which a high-quality moving image can be generated, as compared with a case where the irradiation end timing and the reading start timing are synchronized with each other by using timing devices included in the FPD cassette 3 and the radiation source 2, respectively. In a case where the irradiation end timing and the reading start timing are synchronized with each other by using the timing devices included in the FPD cassette 3 and the radiation source 2, respectively, since the timing devices do not perform time synchronization with each other, a deviation occurs between the timing devices with the passage of time. For example, as a means for correcting the deviation between the timing device of the FPD cassette 3 and the timing device of the radiation source 2, it is conceivable to perform time correction by wireless communication, but since the wireless communication itself is influenced by the surrounding wireless environment, the wireless communication time becomes an uncertain element, and the time cannot be accurately corrected. In the present disclosure, it is possible to perform stable synchronization control without the need for work for correcting a deviation.

As a second effect, the image quality can be improved not only by the determination of the reading start timing by the image controller 223 but also by the automatic exposure control of the radiation source 2 by the output section 222.

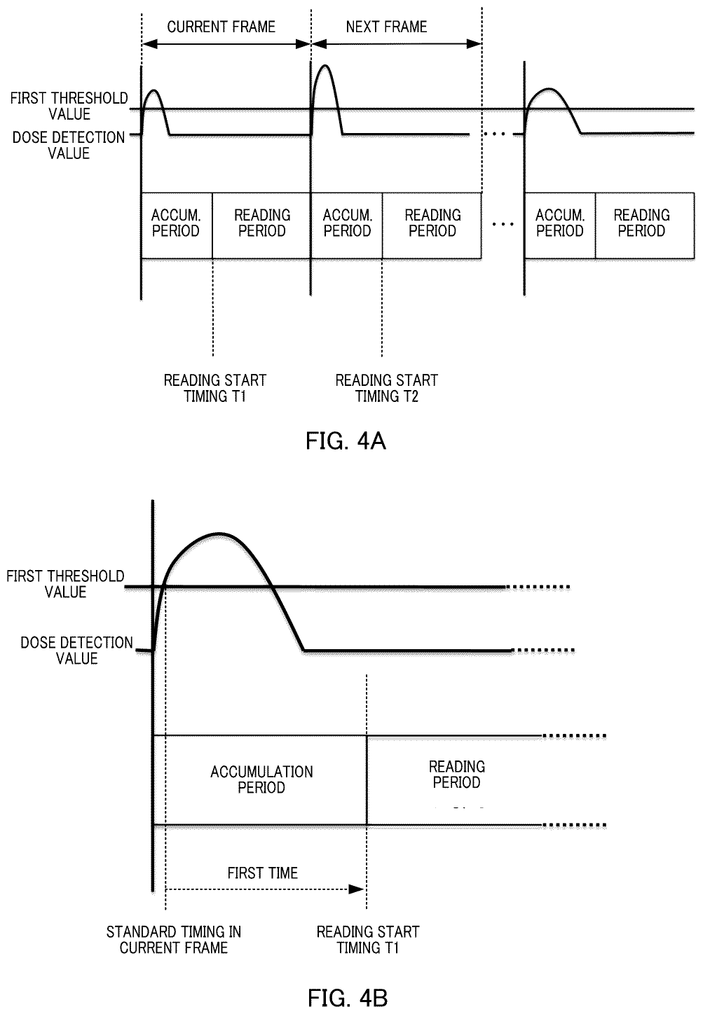

FIGS. 4A and 4B are diagrams for describing a method of determining the reading start timing by the image controller 223. In FIG. 4A, a reading start timing T1 in the current frame and a reading start timing T2 in the next frame are illustrated. In FIG. 4A, “ACCUM. PERIOD” means “ACCUMULATION PERIOD. ” FIG. 4B is an enlarged view of a portion related to the current frame in FIG. 4A. Thus, FIG. 4B illustrates the relationship between the reference timing at which the dose detection value changes from a value equal to or smaller than the first threshold value to a value equal to or larger than the first threshold value and the reading start timing T1 in the current frame. In the present specification, the current frame means a frame based on the radiation related to the dose detection value detected by the dose detection section 221.

The image controller 223 sets, as the reference timing, a timing at which the dose detection value changes from a value equal to or smaller than the first threshold value to a value equal to or larger than the first threshold value in the accumulation period of each frame. Then, the image controller 223 determines the timing at which a first time elapses from the reference timing of the current frame as the reading start timing T1 in the current frame. The first time may be set to an appropriate value based on, for example, a frame rate of a moving image set in advance as an imaging condition. Note that although the image controller 223 determines the reading start timing T1 in the current frame in the examples illustrated in FIG. 4A and FIG. 4B, the present disclosure is not limited thereto. For example, the image controller 223 can determine only the reading start timing of the first frame immediately after the start of irradiation by a different determination method, and determine the reading start timing T2 of the next frame using the reference timing of the current frame as a starting point.

In this way, by appropriately determining at least one of the reading start timings of the current frame and the next frame, it is possible to synchronize the timing of the irradiation of the radiation source 2 and the timing of the accumulation of the FPD cassette 3 with accuracy.

When the reading start timing determined by the above-described method is earlier than the start timing based on the frame rate included in the imaging condition, the image controller 223 may determine the start timing based on the frame rate as the reading start timing. The start timing based on the frame rate included in the imaging condition refers to, for example, a timing obtained by adding a period calculated back from the frame rate, with the reading start timing of the previous frame as a starting point. In this way, it is possible to maintain the frame rate with accuracy while synchronizing the timing of the irradiation of the radiation source 2 and the accumulation of the FPD cassette 3.

Further, when the reading start timing determined by the above-described method is later than the start timing based on the frame rate included in the imaging condition, the image controller 223 may determine the start timing based on the frame rate included in the imaging condition as the reading start timing.

According to these determination methods, it is possible to prevent the generated moving image from becoming a moving image that does not satisfy the condition of the frame rate set as the imaging condition.

Here, as illustrated in FIG. 2, in the FPD cassette 3, the plurality of radiation detection element 7 are arranged in a matrix. The image controller 223 preferably determines the start timing of reading for each of the radiation detection elements 7 so that reading starts sequentially from the radiation detection element 7 arranged at the end portion of the matrix. The reason for this is as follows. Irradiation is continued until an appropriate dose is reached by automatic exposure control, but an irradiation time becomes longer when irradiation is performed with a lower dose. In this case, it is expected that the reading period and the irradiation period overlap each other. Even in this case, when the reading is started sequentially from the end portion of the matrix, the image influence occurs at the end of the image, and the influence on the central portion which is the diagnostic region is reduced.

In the above description, the image controller 223 determines at least one of the reading start timings of the current frame and the next frame. The present disclosure is not limited thereto, and the image controller 223 may determine the read end timing of the current frame and the next frame in addition to the reading start timing or instead of the reading start timing. A method similar to the method of determining the reading start timing may be used as the method of determining the read end timing.

Furthermore, in the above description, the image controller 223 determines the reading start timing for each frame based on the reference timing. For example, instead of acquiring the reference timing in each frame, the image controller 223 may acquire the reference timing once in several frames, for example, and determine each of the reading start timings in the several frames based on the acquired reference timing. Specifically, when the frame in which the reference timing is acquired is set as the first frame, the image controller 223 may determine the reading start timing Tn of the n-th frame by the following Expression (1) (n is an integer of 2 or more).

Tn=P1+Pframe*(n−1) (1)

P1 is the first time, and Pframe is a frame cycle (an inverse of a frame rate, a fixed value).

Since the reference timing is acquired by comparing the dose detection value that rises immediately after the start of irradiation with the first threshold value, the reference timing is easily affected by noise. By acquiring the reference timing only once every several frames, the influence of noise can be minimized.

Furthermore, the image controller 223 may acquire a candidate timing serving as a candidate of the reference timing for each frame by comparing the dose detection value with the first threshold value, and then determine whether to use the candidate timing as the reference timing. Specifically, the following configuration is possible: when the elapsed time from the reference timing of the previous frame to the candidate timing of the current frame is a value greatly different from the frame cycle, the image controller 223 does not use the candidate timing as the reference timing of the current frame. In this case, the image controller 223 may set, as the reference timing of the current frame, a timing at which a frame cycle has elapsed from the reference timing of the previous frame. Also in this case, the influence of noise can be minimized.

Here, the image controller 223 may determine the reference timing of the current frame based on the reference timing of the frame that is two or more frames before the current frame and the frame cycle. For example, data related to reference timings of a plurality of past frames may be stored, and statistical information based on the data may be used to determine whether to use a candidate timing of the current frame as a reference timing. The image controller 223 may derive a time difference between reference timings of adjacent frames as a frame cycle using reference timings of a plurality of past frames, obtain an average and a variation value (variance, standard deviation, or the like) of the plurality of derived frame cycles, and determine whether to use the candidate timing as a reference timing depending on whether an absolute value of a difference between a newly acquired frame cycle and the average frame period exceeds a predetermined value. In this case, the image controller 223 may be configured not to use the candidate timing as the reference timing when the predetermined value is exceeded. As the predetermined value, a fixed value may be used, or a variation value of the frame cycle, for example, a standard deviation σ×0.5 may be used.

Furthermore, in the above description, the image controller 223 directly determines the reading start timing based on the reference timing and the first time period. In the present disclosure, the image controller 223 may indirectly determine the reading start timing instead of directly determining the reading timing as described above. The method of indirectly determining the reading start timing is useful, for example, when another process (e.g., a process for acquiring some correction value) is performed between the start of accumulation and the reading in each frame. This is because there is a case where it is necessary to shift the reading start timing in accordance with the time required for the processing.

Examples of a method in which the image controller 223 indirectly determines the reading start timing include a method in which the end timing of the reading period of a frame immediately before the current frame, the start timing of correction value acquisition, or the start timing or the end timing of the accumulation period before or after the reading period is determined in advance, and the reading start timing is indirectly determined based on the determined timing. Specifically, for example, a timing after the elapse of a certain time from the determined timing may be set as the reference timing. Since the end timing of the reading period of the frame immediately before the current frame, the start timing of correction value acquisition, or the start timing or end timing of an accumulation period before or after the reading period is known at the time of imaging, such a determination method can also determine the reading start timing with accuracy, thus substantially synchronizing the irradiation with radiation and the accumulation period with accuracy.

### Radiation Control Apparatus

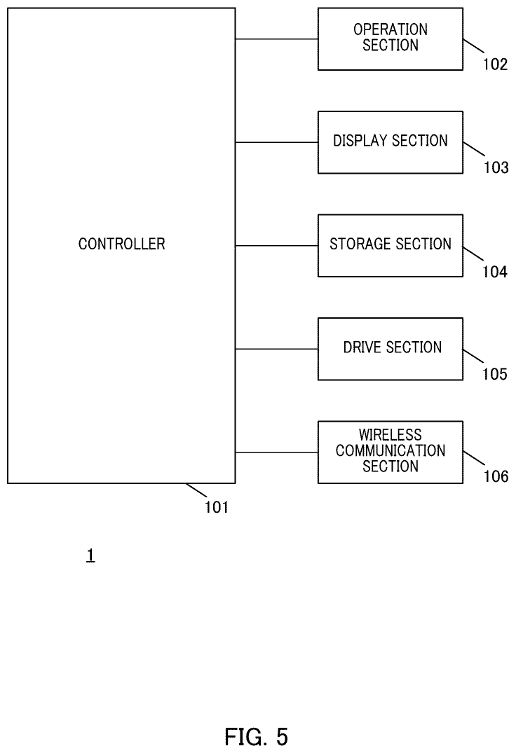

The radiation control apparatus 1 is an apparatus that controls the radiation source 2 and the FPD cassette 3. FIG. 5 is a block diagram illustrating the functional configuration of the radiation control apparatus 1. The radiation control apparatus 1 includes a controller 101, an operation section (operator) 102, a display section 103, a storage section 104, a drive section 105, and a wireless communication section 106.

The controller 101 includes a CPU, a ROM, a RAM, and the like. The CPU of the controller 101 reads a system program and various processing programs stored in the ROM in response to an operation of the operator via the operation section 102, develops the programs in the RAM, and controls the operation of each unit of the radiation control apparatus 1 in accordance with the developed programs. The operator is a person who intends to generate a radiographic image of a subject by using the radiographic imaging system 100, and is, for example, an imaging technician or a doctor.

Examples of the operation section 102 include various buttons, a keyboard, a mouse, a trackball, a touch pad, and the like. The operation section 102 receives an operation of an operator and outputs operation information.

The display section 103 is a display such as a liquid crystal display (LCD), a cathode ray tube (CRT), or an organic EL. The display section 103 is an example of a notification section of the present disclosure. Note that the notification section according to the present disclosure may include not only the display section but also, for example, a sound ringing device such as a speaker or an earphone.

The storage section 104 includes a non-volatile semiconductor memory and a hard disk. The storage section 104 stores programs and parameters necessary for the operation of the components of the radiation control apparatus 1, data related to various processes executed in the radiographic imaging system 100, and the like.

The drive section 105 is a circuit that drives the tube of the radiation source 2. The drive section 105 and the radiation source 2 are connected to each other in a wired manner via, for example, a cable. The wireless communication section 106 performs wireless communication with the FPD cassette 3.

The controller 101 controls the radiation source 2 and the FPD cassette 3 based on imaging condition set in accordance with operator's operation.

The imaging conditions are conditions set by an operator based on a tissue or the like of a subject to be imaged. The imaging conditions are, for example, a radiation dose per unit time and a frame rate of a moving image. The dose of radiation per unit time is, for example, a tube voltage (kV value), a tube current (mA), an irradiation time (sec), or a mAs value. The frame rate of a moving image is a value indicating how many frame images the moving image generated by the radiographic imaging system 100 includes in a unit time (e.g., one second).

### Second Embodiment

Hereinafter, the second embodiment of the present disclosure will be described. In the second embodiment, the cycle at which the dose detection section 221 of the FPD cassette 3 samples the detection value detected by each radiation detection element 7 in the accumulation period can be changed. Except for this point, the configuration and operation of each device of the radiographic imaging system 100 according to the second embodiment are the same as those described in the first embodiment.

When detecting the detection value of the radiation emitted from the radiation source, in a case where the emitted radiation is relatively weak radiation, the ratio of noise included in the detection value detected by the dose detection element increases, when the cycle of sampling the detection value is shortened. This is because, as the detection value per sampling becomes smaller, the number of samplings until the first threshold value for determining the dose detection value is reached increases, and the number of times that noise components are superimposed on the detection value increases. In this case, an error occurs in a detection value detected by the dose detection element and a reference timing for determining a reading start timing is not an accurate timing, which may reduce the accuracy of synchronization between irradiation with radiation and an accumulation period.

On the other hand, conversely, when the emitted radiation is relatively strong radiation, when the cycle of sampling the detection value is lengthened, the amount of change in the detection value per sampling increases, and there is a possibility that the detection value instantaneously exceeds the first threshold value for determining the dose detection value. In this case, an error may occur in the reference timing for determining the reading start timing by the time interval of the sampling period, and the synchronization accuracy may deteriorate.

In the second embodiment, the dose detection section 221 determines whether the radiation emitted by the radiation source 2 is weaker or stronger than a predetermined second threshold value based on the mAs value included in the imaging condition. The second threshold value used for the determination by the dose detection section 221 is a threshold value different from the first threshold value used for the determination of the dose detection value for determining the reading start timing. Next, when the dose detection section 221 determines that the radiation emitted by the radiation source 2 is weaker than the second threshold value, the dose detection section 221 sets the cycle for sampling the detection value to the relatively long first cycle. On the other hand, when the dose detection section 221 determines that the radiation emitted from the radiation source 2 is stronger than the second threshold value, the dose detection section 221 sets the cycle for sampling the detection value to a relatively short second cycle. As a result, appropriate sampling can be performed based on the mAs value set according to the imaging condition, an error in the reference timing for determining the reading start timing can be reduced, and the occurrence of a situation in which the synchronization accuracy decreases can be suppressed.

### Third Embodiment

Hereinafter, the third embodiment of the present disclosure will be described. In the third embodiment, the image controller 223 can determine the reading start timing of the FPD cassette 3 using either one of two different determination methods. Except for this point, the configuration and operation of each device of the radiographic imaging system 100 according to the third embodiment are the same as those described in the first embodiment or the second embodiment.

As described in the first embodiment, the first determination method is a method of determining the reading start timing in the current frame and subsequent frames based on a reference timing determined by the dose detection value.

A second determination method is a method of determining the reading start timing based on the timing device 25 mounted on the FPD cassette 3. In the second determination method, the image controller 223 determines the timing regardless of the dose detection value detected by the dose detection section 221.

In the case of capturing a moving image, the dose of radiation to be emitted each time is much lower than that for capturing a still image. Therefore, in the case of imaging with an extremely low dose, it may be difficult for the dose detection section 221 to detect a dose detection value with accuracy in the accumulation period. When the dose detection value cannot be accurately detected, even when the FPD cassette 3 is controlled at the reading start timing using the first determination method, desired accuracy cannot be obtained for the synchronization between the radiation irradiation and the accumulation period, and a moving image with high image quality cannot be generated in some cases.

Therefore, in the third embodiment, when the mAs value included in the imaging condition is smaller than a predetermined third threshold value (different from the first threshold value and the second threshold value), the image controller 223 determines the reading and accumulation start timing by using the high-precision timing device 25 (see FIG. 2) mounted on the FPD cassette 3. As a result, even when imaging is performed with an extremely low dose of radiation emitted by the radiation source 2, an appropriate method can be selected to synchronize the radiation emission with the accumulation period.

In the third embodiment, the operator may be notified of which determination method is to be used to determine the reading start timing via the display section 103. In the second determination method, it is assumed that a deviation occurs between the timing device 25 of the FPD cassette 3 and the timing device of the radiation source 2 with the passage of time. When the difference exceeds an allowable range, the synchronization between the irradiation with radiation and the accumulation period is broken, and imaging is not established (an image worth diagnosis is not obtained). For this reason, the operator may be notified of the time available for imaging calculated in advance before the start of photographing based on the accuracy of the timer together with the set determination method.

Note that in the third embodiment, at least one of the frame rate and the mAs value may be configured to be changeable before image capturing in response to an operator's operation via an operation section 102 (see FIG. 5) of the radiation control apparatus 1. In this case, a determination method and a time available for imaging in a case where at least one of the frame rate and the mAs value is changed may be notified before image capturing.

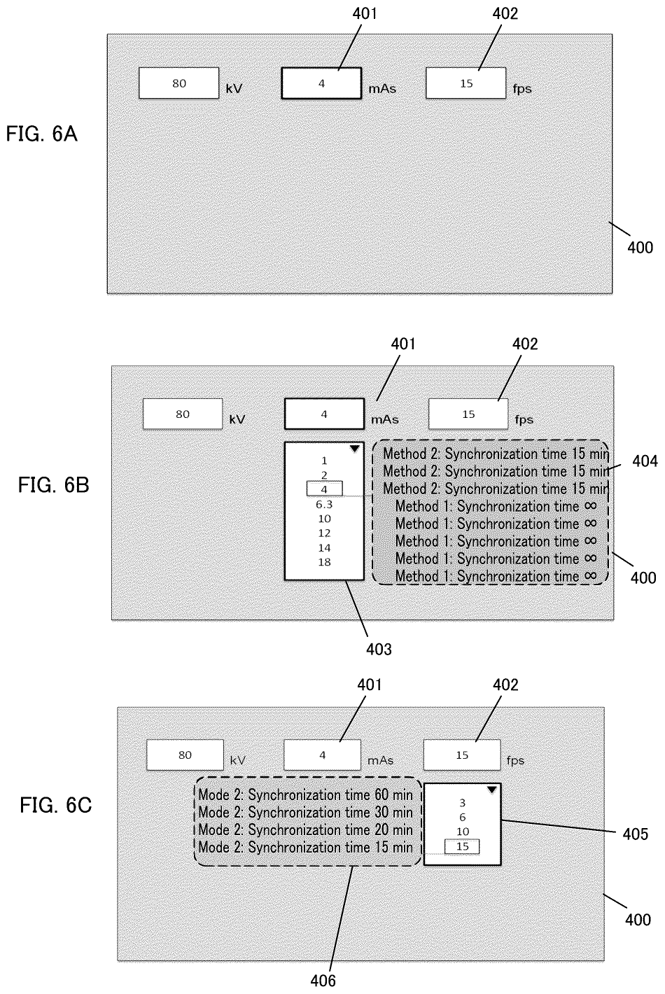

FIGS. 6A to 6C are diagrams illustrating examples of the display screen of the display section 103 in a case where a change in the frame rate or the mAs value is accepted before image capturing. As illustrated in FIG. 6A, a change acceptance screen 400 has an mAs value change field 401 and a frame rate change field 402. In FIGS. 6B and 6C, “Method” means “Determination method” and “Synchronization time” means “Synchronization allowable time. ”

FIG. 6B illustrates an example of a screen displayed when an operator intends to change the mAs value. When the operator selects the mAs value change field 401, an mAs value change candidate field 403 is displayed adjacent to the mAs value change field 401 in the example illustrated in FIG. 6B. In the mAs value change candidate field 403, acceptable candidate value candidates are displayed in a row.

Furthermore, in the example illustrated in FIG. 6B, a determination method suggestion field 404 is displayed adjacent to the mAs value change candidate field 403. In the determination method suggestion field 404, a determination method when each of the change candidate values arranged in the mAs value change candidate field 403 is selected is displayed. In the example illustrated in FIG. 6B, in the determination method suggestion field 404, the time available for imaging in a case where each of the change candidate values listed in the mAs value change candidate field 403 is selected is also displayed together with the determination method. Thus, the operator can easily acquire information necessary for selecting the mAs value.

In the example illustrated in FIG. 6C, an example of a screen when the operator intends to change the frame rate is shown. When the operator selects the frame rate change field 402, in the example illustrated in the 6C of the drawing, a frame rate change candidate field 405 is displayed adjacent to the frame rate change field 402. In the frame rate change candidate field 405, acceptable change value candidates are displayed in a row.

Furthermore, in the example illustrated in FIG. 6C, a determination method suggestion field 406 is displayed adjacent to the frame rate change candidate field 405. In the determination method suggestion field 406, a determination method when each of the change candidate values listed in the frame rate change candidate field 405 is selected is displayed. In the example illustrated in FIG. 6C, in the determination method suggestion field 406, the time available for imaging in a case where each of the change candidate values listed in the frame rate change candidate field 405 is selected is also displayed together with the determination method. Thus, the operator can easily acquire information necessary for selecting the frame rate.

Note that in the present disclosure, the determination method and the notification of the time available for imaging may be provided not only by the display on the display section 103 as in FIGS. 6A to 6C but also by any other method. For example, with a display device in which a plurality of LEDs are arranged in a line, the display may become shorter as the time elapses from the start of imaging and the time available for imaging becomes shorter. In addition, the length of the time available for imaging may be notified by an interval at which the LED indicator blinks. In addition, the time available for imaging may be notified by voice. Such an aspect is useful, for example, when the radiation control apparatus 1 is configured as a medical cart and a space for providing a display device cannot be secured.

While certain embodiments have been described, these embodiments have been presented by way of example only, and are not intended to limit the scope of the invention of the present disclosure. The present disclosure can be implemented in various forms other than the above-described embodiment, and various omissions, substitutions, and changes can be made without departing from the spirit of the invention. The accompanying claims and their equivalents are intended to cover such embodiments or modifications as would fall within the scope and spirit of the invention.

Although embodiments of the present invention have been described and illustrated in detail, the disclosed embodiments are made for purposes of illustration and example only and not limitation. The scope of the present invention should be interpreted by terms of the appended claims.

What is claimed is:

1. A radiographic imaging apparatus that generates a moving image including a plurality of frames by repeatedly executing an accumulation period in which charges based on radiation emitted from a radiation source are accumulated in a plurality of radiation detection elements and a reading period in which reading of the accumulated charges is performed, the radiographic imaging apparatus comprising a hardware processor that executes:
outputting, based on dose information of the emitted radiation, information for controlling emission of the radiation from the radiation source; and
directly or indirectly determining, based on the dose information, at least one of a start timing and an end timing of the reading period in at least one of a current frame and a next frame of the current frame based on the radiation corresponding to the acquired dose information.

2. The radiographic imaging apparatus according to claim 1, wherein
when the determined start timing of the reading period is later than a start timing based on a frame rate set in advance, the hardware processor immediately starts the reading at the start timing based on the frame rate, or the hardware processor starts the reading when the determined start timing of the reading period does not occur within a predetermined time.

3. The radiographic imaging apparatus according to claim 1, wherein
when the determined start timing of the reading period is earlier than a start timing based on a frame rate set in advance, the hardware processor starts the reading at the start timing based on the frame rate.

4. The radiographic imaging apparatus according to claim 1, wherein:
the plurality of radiation detection elements are arranged in a matrix; and
the hardware processor determines at least one of the start timing and the end timing of the reading for each of the plurality of radiation detection elements such that the reading is started sequentially from a radiation detection element of the plurality of radiation detection elements, the radiation detection element being arranged at an end portion of the matrix.

5. The radiographic imaging apparatus according to claim 1, wherein
the hardware processor determines at least one of the start timing and the end timing of the reading period based on the dose information.

6. The radiographic imaging apparatus according to claim 5, wherein
in the accumulation period, the hardware processor outputs, as the dose information, a dose detection value obtained by detecting the radiation emitted to the radiographic imaging apparatus, based on a detection value of at least one of the plurality of radiation detection elements.

7. The radiographic imaging apparatus according to claim 6, wherein
the hardware processor determines at least one of the start timing and the end timing of the reading period based on a reference timing at which the dose detection value changes from a value equal to or smaller than a predetermined first threshold value to a value equal to or larger than the first threshold value.

8. The radiographic imaging apparatus according to claim 7, wherein
the hardware processor generates the dose detection value by sampling the detection value of the radiation at two or more different times within the accumulation period.

9. The radiographic imaging apparatus according to claim 8, wherein
the hardware processor changes a cycle of the sampling of the detection value based on a predetermined second threshold value set in advance.

10. The radiographic imaging apparatus according to claim 9, wherein the first threshold value and the second threshold value are different from each other.

11. The radiographic imaging apparatus according to claim 6, wherein
the hardware processor determines at least one of the start timing and the end timing of the reading period based on an end timing of a reading period of a frame immediately before the current frame, a start timing of correction value acquisition in the current frame, or a start timing or an end timing of an accumulation period before or after the reading period in the current frame.

12. The radiographic imaging apparatus according to claim 5, wherein
based on imaging condition set in advance, the hardware processor sets a method to be used from the following:
a first determination method of determining, based on the dose detection value, at least one of the start timing and the end timing of the reading period in at least one of the current frame and the next frame, or
a second determination method of determining, based on a timing device mounted in the radiographic imaging apparatus, a start timing or an end timing of at least one of the reading period and the accumulation period.

13. The radiographic imaging apparatus according to claim 12, wherein
the imaging condition is a dose per unit time set in the radiation source.

14. The radiographic imaging apparatus according to claim 13, wherein
when the hardware processor determines to use the second determination method, the hardware processor provides notification of a time available for imaging the moving image using the second determination method.

15. The radiographic imaging apparatus according to claim 14, further comprising:
an operator that receives a change in at least one of a frame rate or the dose per unit time, wherein
the hardware processor provides the notification of the time available for imaging corresponding to at least one of the frame rate and the dose per unit time changed in the operator.

16. The radiographic imaging system, comprising:
a radiographic imaging apparatus including a plurality of radiation detection elements;
the radiographic imaging apparatus according to claim 1; and
a radiation source that starts or ends emission of the radiation based on automatic exposure control.
